(19) United States
(12) Patent Application Publication
(10) Pub. No.: US 2009/0012446 A1
(43) Pub. Date: Jan. 8, 2009

(54) DEVICES, SYSTEMS AND METHODS FOR RELEASE OF CHEMICAL AGENTS

(76) Inventors: XINYAN CUI, WEXFORD, PA (US); GUOQIANG BI, PITTSBURGH, PA (US); WILLIAM RICHARD STAUFFER, SLIPPERY ROCK, PA (US)

Correspondence Address: BARTONY & HARE, LLP, 1806 FRICK BUILDING, 437 GRANT STREET, PITTSBURGH, PA, 15219-6101, US

(21) Appl. No.: 12/167,563

(22) Filed: Jul. 3, 2008

Related U.S. Application Data
(60) Provisional application No. 60/958,150, filed on Jul. 3, 2007.

(51) Int. Cl. A61N 1/30; B05D 5/12

(52) U.S. Cl. 604/20; 427/2.1

(57) ABSTRACT

An electrode system includes at least one electrically conductive element to effect at least one of (a) delivery an electrical signal to living cells in the vicinity of the electrically conductive element or (b) measurement of an electrical signal from living cells in the vicinity of the electrically conductive element. The electrically conductive element includes a first conductive polymer which includes at least a first chemical agent to affect activity of the living cells. The first chemical agent is associated with the conductive polymer and is releasable from the first conductive polymer to interact with the living cells upon application of a first electrical potential to the electrically conductive element. As used herein, reference to a first electrical potential includes reference to a range or electrical potentials suitable to effect release of the chemical agent

### CROSS-REFERENCE TO RELATED APPLICATION

This application claims benefit of U.S. Provisional Patent Application Ser. No. 60/958,150, filed Jul. 3, 2007.

### GOVERNMENTAL INTEREST

This invention was made with government support under grant no. 0729869 awarded by the National Science Foundation. The government has certain rights in this invention.

### BACKGROUND OF THE INVENTION

The present invention relates to devices, systems and method for release of chemical agents such as neurochemicals and, particularly, to electrodes including one or more layers of conductive polymer from which one or more neurochemicals can be released.

The following information is provided to assist the reader to understand the invention disclosed below and the environment in which it will typically be used. The terms used herein are not intended to be limited to any particular narrow interpretation unless clearly stated otherwise in this document. References set forth herein may facilitate understanding of the present invention or the background of the present invention. The disclosure of all references cited herein are incorporated by reference.

Fundamental brain functions such as cognition, learning, and memory are carried out by the coordinated activity of many neurons that form intricate circuits. Recent advances in computer, electronics and fabrication technologies have led to the development of various multi-electrode arrays (MEAs) for the study of ensemble neural activity at the circuit level both in vitro and in vivo by simultaneous extracellular recording (and sometimes stimulating) of neuronal activity at many different locations. Typical in vitro MEAs have patterned microelectrodes and conductive leads embedded in the center of a glass plate on which dissociated cells, acute slices, or organotypic slices are placed and grown. FIGS. 1A and 1B display an example of an MED64 System probe (available from Panasonic) on which 64 planar microelectrodes are patterned. The conductive leads are made of indium tin oxide or ITO, and platinum black is deposited on the electrode regions to lower the impedance for recording and stimulation.

For in vivo studies, the majority of MEAs are silicon based and are implanted into the brain. FIGS. 2A and 2B show examples of an Acute Probe (available from NeuroNexus Technology of Ann Arbor, Mich.). In these electrodes, a micromachined silicon substrate supports an array of thin-film conductors that are insulated above and below by deposited silicon dioxide dielectric. Openings in the dielectric at the probe tip regions define the vertical connection to stimulating or recording sites that are sputtered with gold or iridium for interfacing with the tissue. These neural probes have been successfully used in neural stimulation and single unit recording from various brain regions.

Because of the chemical nature of many neuronal signaling processes, including synaptic transmission and modulation, it is often necessary to perturb neurons and circuits with pharmacological manipulations. Consequently, combining pharmacological manipulations with electrophysiological recording is often a preferred powerful approach to dissect the cellular mechanisms of neural activity and modulation as well as the neuronal basis of system behavior.

Conventionally, global perfusion, systematic administration and intracortical injection have been the most commonly used methods of drug delivery in vitro and in vivo for studies that require low spatial and temporal resolution. Other methods such as pressure puffing and iontophoresis through micropipettes have been employed for local delivery. These methods are generally limited to a few target sites. Recent efforts have been made to fabricate microelectrodes with fluid channels, which eliminates the requirement of extra positioning devices and improves the spatial resolution of local delivery. However, the addition of channels, pumps, and valves for active delivery control complicates device fabrication, and can lead to lower yield, higher cost and higher failure rate. Furthermore, the size of the device will be increased, which is undesirable for minimizing insertion injury and host tissue reaction for in vivo applications.

Recently, conductive polymers have been explored for use in drug delivery. Conductive polymers require ionic dopants to conduct electric currents. It is known that some conductive polymers can undergo electrically controllable, reversible redox reactions, which involve the charging and discharging of the polymer and are accompanied by the movement of hydrated ions. Utilizing this feature, efforts have been made to incorporate charged drug molecules into conductive polymers. The drugs molecules are released in response to an electrical stimulus. In several prototypic systems, chemical agents such as anionic salicylate, adenosine triphosphate (ATP), 2-ethylhexylphosphate (EHP), naproxen and cationic dopamine have been incorporated into different polymers and subsequently released in a controlled fashion. It has also been shown that dexamethasone sodium phosphate (Dex) can be incorporated into polypyrrole (PPy) or poly(3,4-ethylenedioxythiophene) (PEDOT) film coating a gold electrode and released in response to a cyclic voltammetric (CV) stimulus. Wadhwa, R., Lagenaur, C. F. & Cui, X. T. Electrochemically controlled release of dexamethasone from conducting polymer polypyrrole coated electrode. Journal of Controlled Release 110, 531-541 (2006).

It is desirable to develop improved devices, systems and methods for delivery of chemical agents such as neurochemical agents in connection with, for example, biological electrode function (for example, neuronal electrode function).

### SUMMARY OF THE INVENTION

In one aspect, the present invention provides an electrode system including at least one electrically conductive element to effect at least one of (a) delivery an electrical signal to living cells in the vicinity of the electrically conductive element or (b) measurement of an electrical signal from living cells in the vicinity of the electrically conductive element. The electrically conductive element includes a first conductive polymer which includes at least a first chemical agent to affect activity of the living cells. The first chemical agent is associated with the conductive polymer and is releasable from the first conductive polymer to interact with the living cells upon application of a first electrical potential to the electrically conductive element. As used herein, reference to a first electrical potential includes reference to a range or electrical potentials suitable to effect release of the chemical agent.

The chemical agent, including neurochemicals, of the present invention can, for example, be repeatably releasable from the first conductive polymer to interact with the living cells upon cyclic application of the first electrical potential to the electrically conductive element.

The first conductive polymer can, for example, be polypyrole, poly(3,4-ethylenedioxythiophene), polyaniline, polythiophene, poly(acetylene), poly(fluorene), polytetrathiafulvalene, polynaphthalene, poly(p-phenylene sulfide), poly(para-phenylene vinylene), a derivative of the above, a copolymer of the above, or a mixture of the above. In several embodiments, the first conductive polymer is deposited upon the electrically conductive element via electropolymerization.

In several embodiments, the living cells are neuronal cells and the first chemical agent is a first neurochemical. The first neurochemical can, for example, be a neurotransmitter, a neuromodulator, an agonist or a receptor antagonist. Examples of suitable neurochemicals include, but are not limited to 6-cyano-7-nitroquinoxaline-2,3-dione, 2-amino-5-phosphonopentanoic acid, glutamate, acetylcholine, epinephrine, norepinephrine, dopamine, serotonin and melatonin, glutamic acid, gamma aminobutyric acid (GABA), aspartic acid, glycine, adenosine, adenosine-5′-triphosphate (ATP), guanosine triphosphate (GTP), bicuculin, dizocilpine (MK801), or a derivative of one of the above.

The electrode system can, for example, include a plurality of conductive electrode elements wherein at least two of the conductive electrode elements include the first conductive polymer and the first neurochemical.

In several embodiments, the electrode system includes at least a second conductive electrode element including a second conductive polymer. The second conductive polymer can include a second neurochemical that is releasable upon application of a second potential to the second conductive electrode. In a number of embodiments, the first conductive polymer and the second conductive polymer can be the same, the first neurochemical and the second neurochemical can be the same, and the first potential and the second potential can be the same. The first neurochemical and the second neurochemical can also be different. The first conductive polymer and the second conductive polymer can likewise be different. Further, the first potential and the second potential can be different.

The second conductive polymer can, for example, be polypyrole, poly(3,4-ethylenedioxythiophene), polyaniline, polythiophene, poly(acetylene), poly(fluorene), polytetrathiafulvalene, polynaphthalene, poly(p-phenylene sulfide), poly(para-phenylene vinylene), a derivative of the above, a copolymer of the above, or a mixture of the above. In several embodiments, the first conductive polymer is deposited upon the electrically conductive element via electropolymerization.

The second neurochemical can, for example, be 6-cyano-7-nitroquinoxaline-2,3-dione, 2-amino-5-phosphonopentanoic acid, glutamate, acetylcholine, epinephrine, norepinephrine, dopamine, serotonin and melatonin, glutamic acid, gamma aminobutyric acid (GABA), aspartic acid, glycine, adenosine, adenosine-5′-triphosphate (ATP), guanosine triphosphate (GTP), bicuculin, dizocilpine (MK801), or a derivative of one of the above.

The electrode system can for example be a multielectrode array including a plurality of electrically conductive microelectrode elements. The electrode system can also include a single conductive microelectrode element.

The first conductive polymer and/or the second conductive polymer can, for example, be selectively deposited upon electrically conductive elements of the multielectrode (or other electrode) array via electropolymerization.

In another aspect, the present invention provides a method of applying a chemical agent to living cells, including: contacting at least one electrically conductive element of an electrode system with the living cells, the electrically conductive element being adapted to effect at least one of (a) delivery an electrical signal to living cells in the vicinity of the electrically conductive element or (b) measurement of an electrical signal from living cells in the vicinity of the electrically conductive element, the electrically conductive element including a first conductive polymer, the first conductive polymer including at least a first chemical agent to affect activity of the living cells, and causing the first chemical agent to be released from the first conductive polymer to interact with the living cells by application of a first electrical potential to the electrically conductive element. The first chemical agent can, for example, be a first neurochemical as described above.

In another aspect, the present invention provides a method of fabricating an electrode system including at least one electrically conductive element to effect at least one of (a) delivery an electrical signal to living cells in the vicinity of the electrically conductive element or (b) measurement of an electrical signal from living cells in the vicinity of the electrically conductive element, including: applying at least a first conductive polymer, the first conductive polymer to the electrically conductive element, and doping the first conductive polymer with at least a first chemical agent to affect activity of the living cells, the first chemical agent being releasable from the first conductive polymer to interact with the living cells upon application of a first electrical potential to the electrically conductive element. The conductive polymer can be doped either before or after applying or depositing the conductive polymer on the electrically conductive element.

Based on the drug releasing properties of conducting polymers, the devices, systems and methods of the present invention provide targeted delivery of small amounts of chemical agents (such as neurochemicals) in real time that can be easily integrated into pre-existing in vitro and in vivo electrodes such as multielectrode arrays used in connection with living cells (such as neurons) without complex microfabrication. The inventors have observed that chemical agent-doped conductive polymer coatings do not interfere with the recording and/or stimulating capability of the electrodes. Indeed, in many cases, the chemical-agent-doped conductive polymers substantially improve the electrical properties of microelectrodes. Conventional electrodes, including multielectrode arrays, can be selectively modified with a one or more specific polymer coatings, thus becoming capable of both electrical signal recording/stimulus and controlled drug release of one or more chemical agents. For example, the devices, systems and methods of the present invention allow electrode arrays to directly and repeatedly deliver a combination of different chemical agents at small, controlled quantities (for example, on the order of femtogram or 10⁻¹⁵ grams) to local targets (for example, within hundreds of micrometers distance) in real time, while maintaining their electrical recording/stimulating capability. The present invention thus provide a powerful tool in, for example, manipulating neuronal properties at specific local circuits with previously unattainable spatial and temporal precision.

Once again, the conductive polymer-based drug release systems of the present invention are readily incorporated upon existing electrodes such as microelectrode arrays or MEAs. In general, microelectrodes have dimensions less than 1 mm and typically have dimension in the range of 10 to 100 μm or even less. In several embodiments, the coating, layer or film of the doped conductive polymers of the present invention deposited upon the electrode(s) is relatively thin. For example, in several embodiment, the coating is less than 50 μm or even less than 10 μm. Electrodes and electrode arrays of the present invention enable direct and repeated delivery of one or more chemical agents. The devices, systems and methods of the present invention are applicable to many chemical agents (for example, neurochemicals such as neurotransmitters, neuromodulators, agonists and antagonists) and can be extended to, for example, various neural and other electrodes.

The controllability and spatiotemporal resolution of the devices, systems and methods of the present invention allow for dissecting molecular, neuronal, and circuit functions with high precision, and for investigating a wide range of challenging problems that are otherwise difficult to address. Combining chemical manipulations with multi-electrode neural recording can, for example, be a powerful approach to dissect the cellular mechanisms of neural activity and modulation as well as the neuronal basis of system behavior. The present invention allows one to experimentally test a wide range of challenging hypotheses regarding the dynamics and function of neuronal circuits. For example, does the activation of certain receptors (e.g. NMDA receptors) underlie a particular activity pattern (e.g. reverberation) in a specific cortical circuit? Which neural modulator (e.g., dopamine or acetylcholine) at what time enhances the plasticity of specific neurons and circuits? Is the activity of a circuit at a given time important for certain cognitive behavior (e.g. decision making)?

In addition to research, the devices, systems and methods of the present invention can also have therapeutic applications in the diagnosis and treatment procedures (for example, diagnosis and/or treatment of some neurological disorders such as epilepsy and Parkinson's diseases etc.). For example, a precursor state of a seizure episode can be detected using an implanted microelectrode array of the present invention, and a chemical agent can be delivered using the conductive polymer-chemical agent coating of the microelectrode array to reduce the severity of or to prevent the seizure episode.

The present invention, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates an MED64 multielectrode array probe currently available from Panasonic.

FIG. 1B illustrates the electrode arrangement of the multielectrode array of FIG. 1A.

FIG. 2A illustrates an embodiment if an Acute Probe multielectrode array (available from NeuroNexus Technologies of Ann Arbor, Mich.).

FIG. 2B illustrates a probe mounted to a PC board and ready for in vivo implantation.

FIG. 3A illustrates an example of a trace of polysynaptic current under control conditions (ctrl).

FIG. 3B illustrates an example of a trace of polysynaptic current with 25 μM D-AP5 (AP5).

FIG. 3C illustrates an example of a trace of polysynaptic current under conditions of wash with recording solution that soaked the macroelectrode without voltage pulses (wash1).

FIG. 3D illustrates an example of a trace of polysynaptic current under conditions of wash with recording solution containing electrically released AP5 (AP5_r).

FIG. 3E illustrates an example of a trace of polysynaptic current under conditions of wash with normal recording solution (wash2).

FIG. 4 illustrates a portion of an MED64 chip showing selective coating with different compositions of conducting polymer/dopant (wherein coated microelectrodes are cross-hatched)

FIG. 5A illustrates voltage traces from a single neuron show the suppression of neuronal spiking after release of CNQX from the recording electrode and subsequent recovery.

FIG. 5B(i) illustrates a post-stimulus or peristimulus time histogram (PSTH) derived from the raster plots using 1 s. time bins.

FIG. 5B(ii) illustrates a raster plot of repeated trials from a single cell recorded from the CNQX-releasing electrode for eight trials.

FIG. 5C(i) illustrates two simultaneous 1 minute voltage profiles from electrodes a and b, respectively, before CNQX release.

FIG. 5C(ii) illustrates two simultaneous 1 minute voltage recordings of the same 2 electrodes after CNQX was released from electrode a.

FIG. 5D illustrates a PSTH derived from the firing of three different cells recorded from non-releasing electrodes during the drug release trials detailed in FIG. 5B.

FIG. 5E illustrates a voltage trace of showing neuronal activity on an electrode not coated with the CNQX-releasing polymer before and after the current stimulus was applied.

FIGS. 6A illustrates a photomicrograph image of a low-density neuronal network growing on a conducting polymer modified multielectrode array (MED64 available from Panasonic).

FIG. 6B illustrates graphically an experimental protocol used in connection with the multielectrode array of FIG. 6A wherein neural stimulations (ns1,2, . . . ,8) are delivered through the substrate electrodes but not through the releasing electrode.

FIG. 6C illustrates eight columns corresponding to ns1 . . . 8, and six rows corresponding to individual trials and a bar graph comparing the normalized peaks of the adjusted currents for the CNQX release (cross-hatched) and control release at each time-point.

FIG. 6D illustrates the effect of CNQX release across all trials, wherein the data is collapsed in time.

FIG. 6E illustrates that the distance of the cell to the releasing electrode has a significant effect.

FIG. 7 illustrates an impedance spectroscopy study of conducting polymer poly(3,4-ethylenedioxythiophene) or PEDOT coated electrodes on an Acute Probe available from NeuroNexus Technology and an image of a coated probe shank with deposition charge indicated for each electrode.

FIG. 8 illustrates various electrical release stimuli for use in connection with electrodes of the present invention.

### DETAILED DESCRIPTION OF THE INVENTION

As used herein and in the appended claims, the singular forms “a,” “an”, and “the” include plural references unless the content clearly dictates otherwise. Thus, for example, reference to “a conductive polymer” includes a plurality of such conductive polymers and equivalents thereof known to those skilled in the art, and so forth, and reference to “the conductive polymer” is a reference to one or more such conductive polymers and equivalents thereof known to those skilled in the art, and so forth.

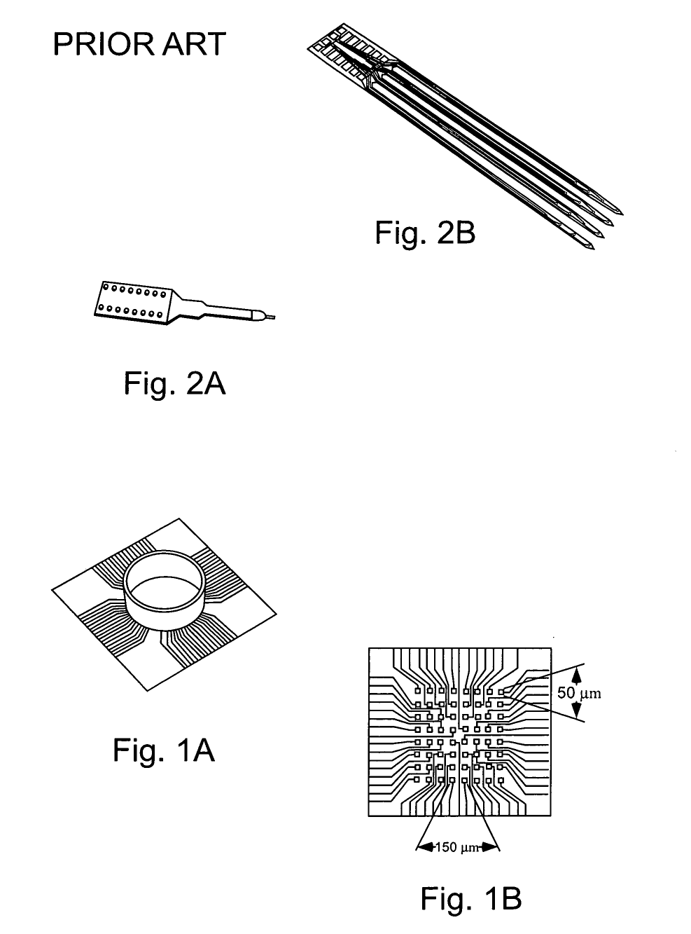

In several studies of the present invention, selected neurochemicals together with conductive polymers are incorporated onto an in vitro multielectrode array or MEA system (such as the MED64 System illustrated in FIGS. 1A and 1B). The release properties of the neurochemicals were studied using cultured rat neurons as sensors. Loading capacity, release rate, and diffusion range of the drug molecules and their effectiveness on modulating local neuronal networks in vitro are readily studied. Using essentially the same procedures, drug-polymer complexes can also be incorporated onto an in vivo MEA systems such as illustrated in FIG. 2A and 2B.

For interfacing neurons with electrically conductive polymer electrodes, we have used an electropolymerization method to directly deposit a conducting polymer/dopant complex on an electrode surface as shown below, using, for example, the conducting polymer polypyrrole as a representative example.

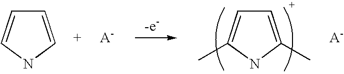

In the above formula, A⁻ represents an anion dopant. As the monomers grow into polymers and are deposited onto the anode, anions in the solution will be incorporated. This is a convenient mechanism for incorporating bioactive molecules/chemical agents onto the conducting polymer electrode. These bioactive molecules can be immobilized to encourage specific cell/surface interaction, or releasable to regulate the local biochemical environment. The chemical agent(s) can, for example, be loaded during the electropolymerization or after polymerization when the polymer is soaked in drug solution under negative electrical potential

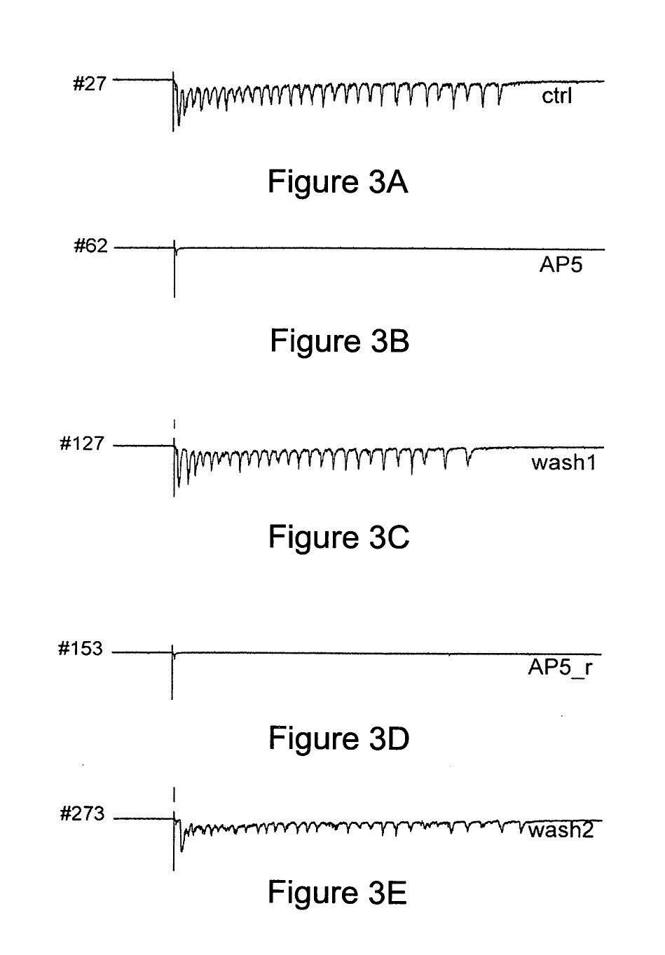

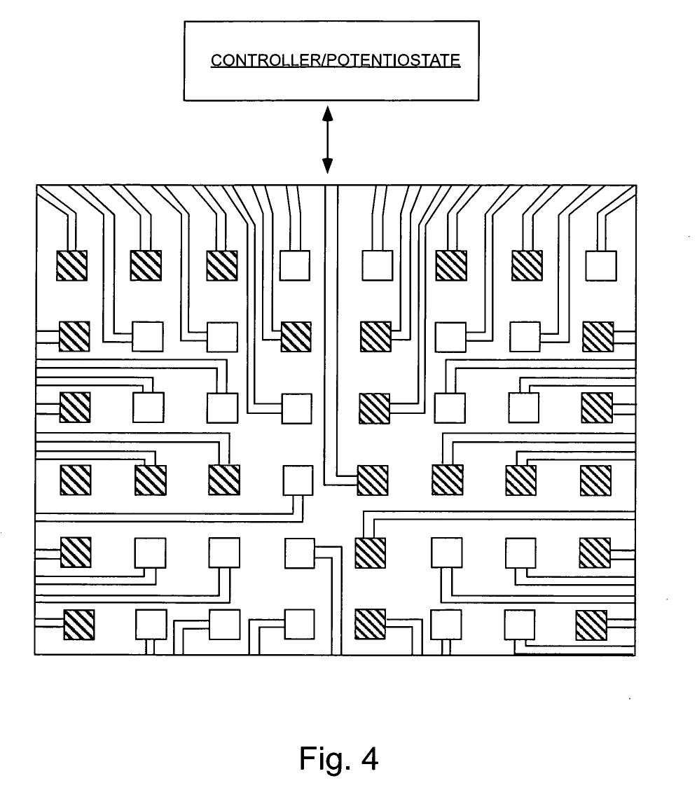

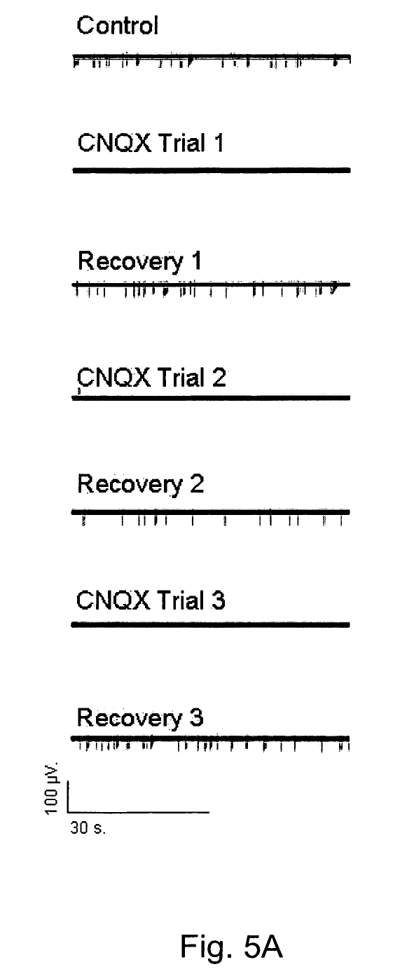

In several studies, the NMDA-type glutamate receptor antagonist AP-5 was, for example, incorporated as a dopant into PPy-coated gold “macro-electrodes” (having an area of 0.77-cm²). In general, macro-electrodes or macroelectrodes have dimensions greater than 1 mm. A hippocampal neuronal culture and patch clamping were used to test the effectiveness of AP5 release. The drug was released from the electrode into normal recording solution by applying 30 cyclic voltage stimuli. This solution, when added to the neuronal culture, blocked evoked reverberatory activity in the network. Released AP5 from the polymer effectively suppressed network reverberation. 280 trials (at 30 s intervals) of evoked activity of a cultured hippocampal network under 5 different conditions were made in several studies as follows: 1. control (ctrl); 2. with 25 μM D-AP5 (AP5); 3. wash with recording solution that soaked the macroelectrode without voltage pulses (wash1); 4. with recording solution containing electrically released AP5 (AP5_r); 5. wash with normal recording solution (wash2). Polysynaptic current traces with color coded current amplitude (in nA) indicated that reverberatory activity normally seen in ctrl and wash conditions was completely blocked by added AP5 and by AP5 released from the macroelectrode. FIGS. 3A-3E sets forth example traces of polysynaptic current with one sample trace for each test condition. The scale bar in FIGS. 3A-E is 0.5 nA, 1 s.

We estimated that the amount of AP5 released from one CV stimulus is ˜2 μg/cm². Furthermore, after 30 CV stimuli, the film remained microscopically intact suggesting a relatively long lifetime of the drug releasing film. If the same polymerization condition is preserved for microelectrodes, a shorter command pulse could release enough AP5 to reach an effective concentration in the vicinity of a 50×50 μm² electrode.

Coating multielectrode arrays through electropolymerization provides substantial flexibility. Because the electrodes in the arrays are individually addressable, one can selectively coat each electrode with one or more conductive polymers, and easily incorporate different drugs onto different electrodes on the same array. In several studies, an in vitro multichannel neural recording system was created. The test system also includes a hardware connection that links the multielectrode array (see FIG. 1B) chip to a potentiostat for performing electrode coating deposition, impedance spectroscopy and cyclic voltammetry operations directly on each individual microelectrode. Using this system, we have succeeded in selectively coating microelectrodes with conducting polymer loaded with various dopant molecules for controlled release For better demonstration of the black conducting polymer coating, transparent ITO electrodes were used. For all other experiments, we used platinum black electrodes. The coated electrodes recorded quality signals of spontaneous neural activity from cultured neurons. FIG. 4 illustrates a portion of an MED64 chip showing selective coating with different compositions of conducting polymer/dopant (wherein coated microelectrodes are cross-hatched). The size of each microelectrode is 50×50 μm².

CNQX is an AMPA type glutamate receptor antagonist which inhibits excitatory neural transmission. We electrodeposited polypyrrole/CNQX on the microelectrodes of MED64 probes which were than plated with neuronal cultures from E18 rat cortices. When spontaneous activity was observed on an electrode bearing a CNQX/PPy film, a current stimulus (four 1 s-long pulses of −5 μA with 200 ms refractory periods in between) was applied to the electrode. Recordings were taken before and after the stimulus to observe any inhibition and recovery (during the stimulation it was not possible to record from the particular electrode given the pulse).

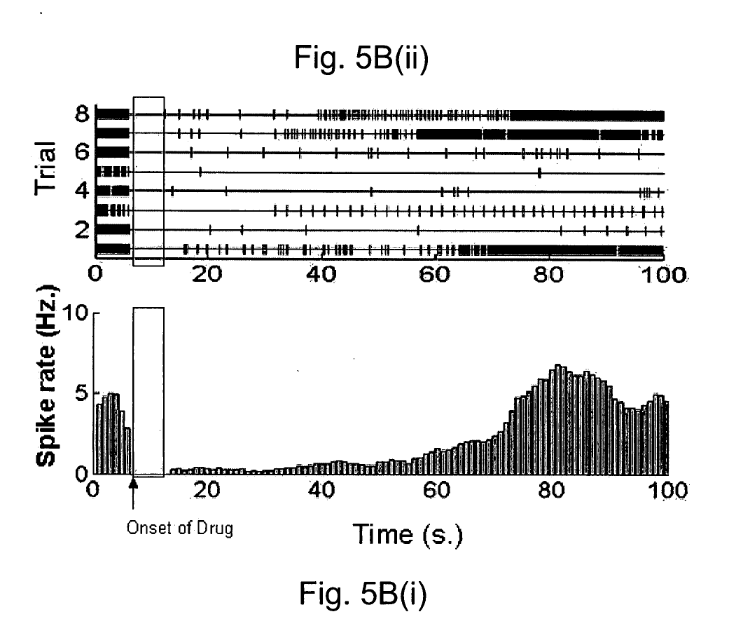

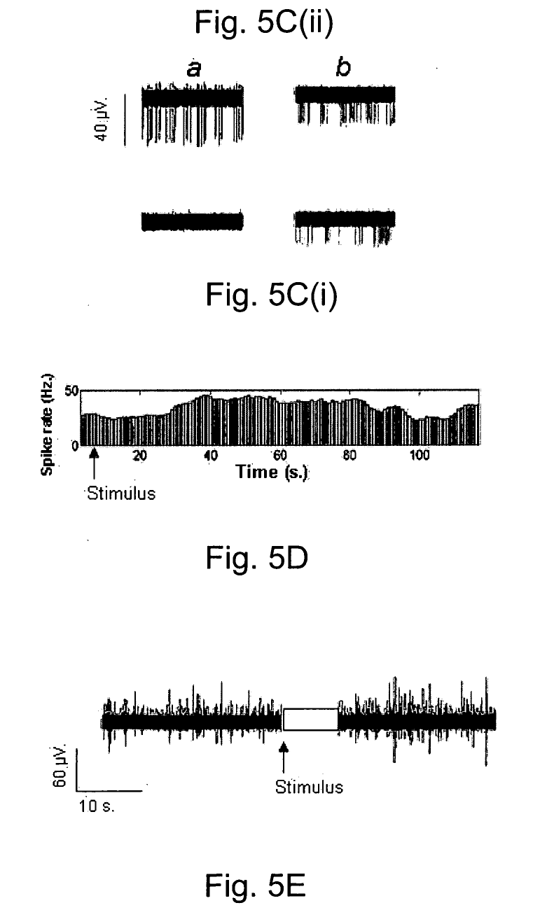

The results are summarized in FIGS. 5A through 5E. It was observed that neural spiking disappeared after applying the stimulus to an electrode bearing the PPy/CNQX. And at a later time point action potentials from the same cell (determined by examination of the waveforms in Offline Sorter and Wave Tracker, Plexon Inc.) reappeared on the electrode and the rate returned to baseline (determined by pre-stimulus control recordings). This effect could be repeatedly induced by subsequent CNQX release on an individual cell a number of times (see FIG. 5A). FIG. 5A illustrates voltage traces from a single neuron show the suppression of neuronal spiking after release of CNQX from the recording electrode and subsequent recovery. The seven trials were performed in the order shown to demonstrate the transient and repeatable nature of the CNQX delivery method and response. We determined, through repeated release, inhibition and recovery cycles, a time course of inhibition and recovery (see FIG. 5B) that appears consistent with the published effect of CNQX and with the theoretical estimate of CNQX diffusion in solution. In the studies of FIG. 5B, repeated CNQX release trials were performed to determine a time course of recovery. The top plot in FIG. 5B shows the raster plots of a single cell recorded from the CNQX-releasing electrode for the first eight trials. The second plot in FIG. 5B is the PSTH derived from the raster plots using 1 s. time bins. The recording apparatus did not permit simultaneous stimulation and recording from the same electrode. The electric current stimulation used to release the CNQX had a 4 s. duration and the integrated amplifier had an approximate 1 s. recovery time after switching from stimulation to recording. This “quiet time” is denoted by the shaded block. After eight trials, the inhibitory effect diminished, possibly because of exhaustion of the CNQX supply. The inhibition of spiking activity on an electrode did not affect spiking activity on neighboring electrodes (see FIGS. 5C and D). The observable effect of CNQX was highly localized, cell a and cell b were recorded simultaneously on two different electrodes with an inter-electrode distance of 212 μm. The top row of FIG. 5C shows two simultaneous 1 minute voltage profiles from cells a and b, respectively, before CNQX release. The bottom row of FIG. 5C shows two simultaneous 1 minute voltage recordings of the same 2 cells after CNQX was released from electrode a. In FIG. 5D, PSTH was derived from the firing of three different cells recorded from non-releasing electrodes during the drug release trials detailed in FIG. 5B. The time of CNQX release is shown by the arrow. The electrical stimulus itself does not cause any transient inhibition of neural activity. As shown in FIG. 5E, the effect of the stimulation on neuronal firing was negligible. The voltage trace of FIG. 5E shows neuronal activity on an electrode not coated with the CNQX-releasing polymer before and after the current stimulus was applied. As described in connection with FIGS. 5A and 5B “quiet time” is denoted by the shaded block. The results of these studies show that it is possible to deliver neurochemicals directly from the recording electrodes and the delivery is precisely controlled by the electrical stimuli (in the range of, for example, femtograms per stimuli) and the effect of the drug can be localized to a distance on the order of hundreds of micrometers.

The above studies of CNQX and AP-5 release demonstrate the local release of neurochemical to influence neurotransmission. As the drug loading and release mechanisms described herein are common to many molecules, the devices, systems and methods of the present invention can be used in connection with many chemical agents, including many neurochemicals. Different drug may require different loading and releasing schemes, and our multi-step approach should lead to optimized conditions.

Drug release and diffusion properties can, for example, be studied with low-density culture and patch-clamp recordings. The range and duration of drug effect also reflect properties of network architecture and dynamics. By transiently perturbing local neuronal activity, the systems of the present invention can provide better inference of network connectivity by providing a mechanism to test hypothetical network connections found through traditional correlation of spiking activity. In addition, such experiments can reveal how local circuit modulation affects global network dynamics.

A limitation the MED64 system described above is that recording can not be done simultaneously during stimulation used for triggering the drug release. There is a 2-second delay between the end of stimulation and beginning of recording when stimulation artifact dominates. This limitation is not a problem given the prolonged drug effect observed in the above studies. However, for applications requiring fast excitation or inhibition, the drug effect may be embedded in the “delay” period. Patch clamp experiments can provide a good indication of whether this is the case or not. Moreover, if an immediate and short time course (less than 2 s.) for observing drug effect is necessary for certain applications, hardware is available that enables almost simultaneous stimulation and recording. See, for example, Wagenaar, D. A. and Potter, S. M. A versatile all-channel stimulator for electrode arrays, with real-time control. Journal of Neural Engineering 1: 39-45 (2004).

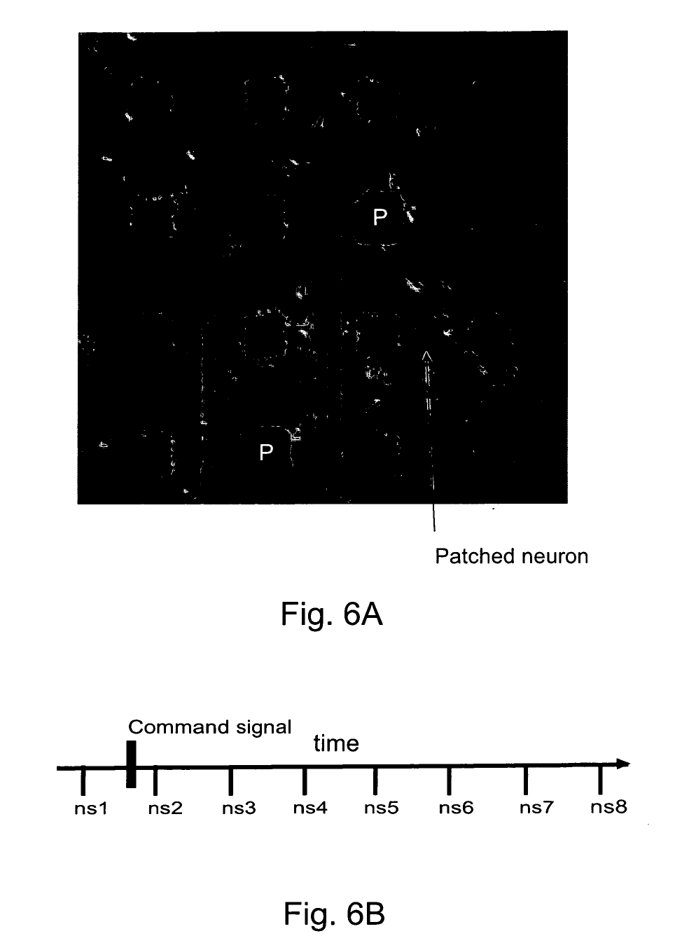

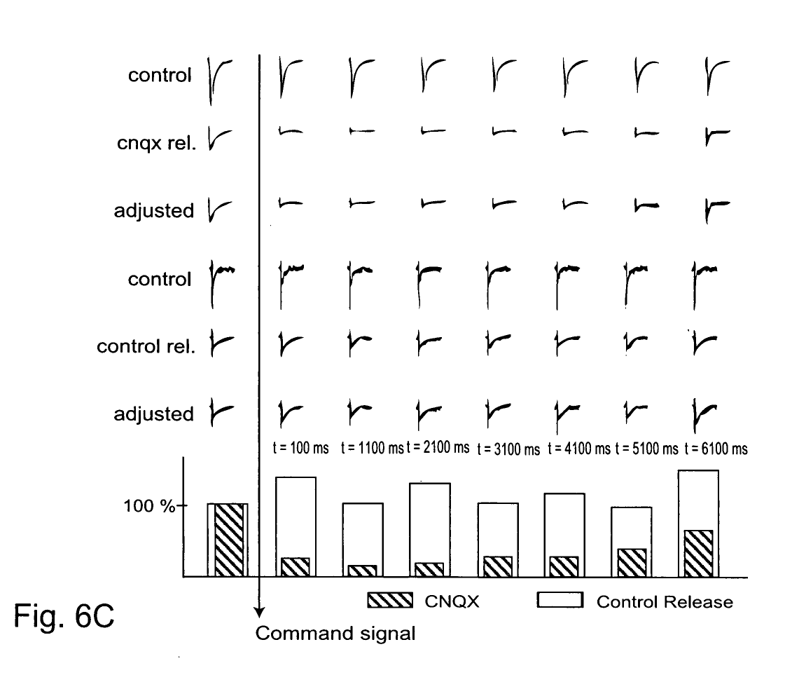

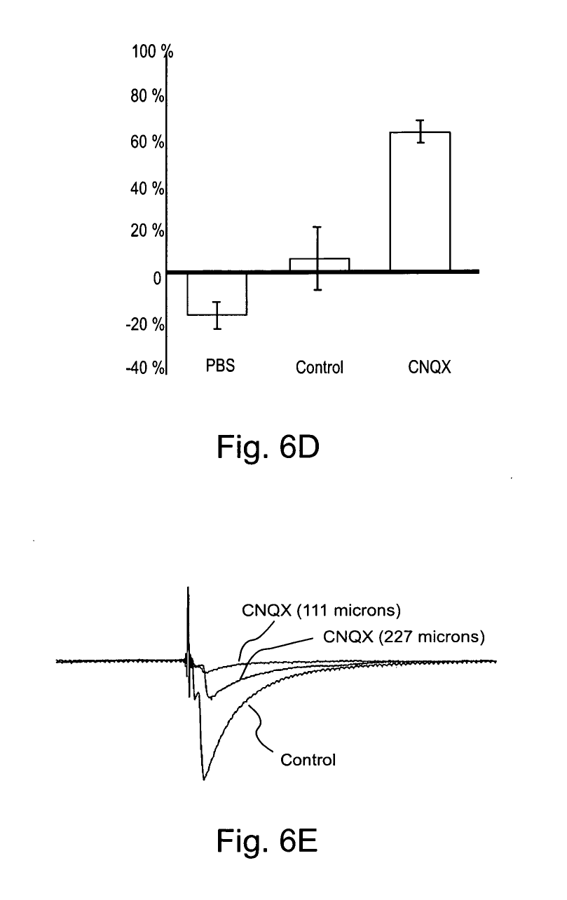

FIG. 6A is a photomicrograph image of a low-density neuronal network growing on a conducting polymer modified multielectrode array (MED64 available from Panasonic). The platinum coated electrodes are modified with the conducting polymer poly-pyrrole (PPy) containing either CNQX or chloride and phosphate ions (P) from PBS (blank release control). Release of the drug is affected by a “command signal,” which is the delivery of a voltage pulse (−2.5 V, 200 ms). Neuron “firing,” the generation of action potentials, is an all or none phenomenon. Synaptic currents are a continuous and graded phenomenon. For this reason synaptic currents recorded by patch electrodes better reflect the release and diffusion of the synaptic inhibitor CNQX, compared to action potentials recorded by the extracellular electrodes. To monitor the effect of release, individual cells in the network are patch clamped (wherein the patched cell is indicated by an arrow in FIG. 6A). The experimental protocol is described graphically in FIG. 6B. Neural stimulations (ns1,2, . . . ,8) are delivered through the substrate electrodes (but not the releasing electrode). The command signal starts 300 ms prior to ns2, and the currents are recorded by the patch electrode. Following the command signal to an electrode with a PPy film containing CNQX, there is a significant decrease in the size of monosynaptic currents. FIG. 6C sets forth 8 columns corresponding to ns1 . . . 8, and 6 rows corresponding to individual trials. The first three rows correspond to data from one cell (close to a CNQX containing electrode) and the final three rows show data from a different cell (close to an electrode not containing CNQX). The rows labeled “control” show the results of 8 neural stimuli (100 μs, 1 V). The small decrease in the size of the currents is probably a result of short-term depression. The current is almost completely abolished following CNQX release. The third row, labeled “adjusted,” takes into account the short-term depression seen in the control trial by dividing each point in the release trials by the ratio of the peak of the control response at the corresponding neural stimulation and the peak of the baseline current. Rows 3 through 6 repeat the process above but with data obtained after the command signal was delivered to a PBS modified electrode. The bar graph at the bottom of FIG. 6C compares the normalized peaks of the adjusted currents for the CNQX release (cross-hatched) and control release at each time-point. Examination of this graph shows that the neuron begins to recover towards the end of the trial. Across all trials, and by collapsing the data in time, FIG. 6D demonstrates a significant effect of CNQX release. Furthermore, as shown in FIG. 6E, the distance of the cell to the releasing electrode has a significant effect. This effect is expected from the proposed model of release and diffusion of CNQX. The trace labeled CNQX (227 microns) shows the average of 8 trials after release of CNQX from an electrode located 227 μm distant from the cell. The trace labeled CNQX (111 microns) is the average of 8 trials from the same cell but a different electrode located 111 μm distant.

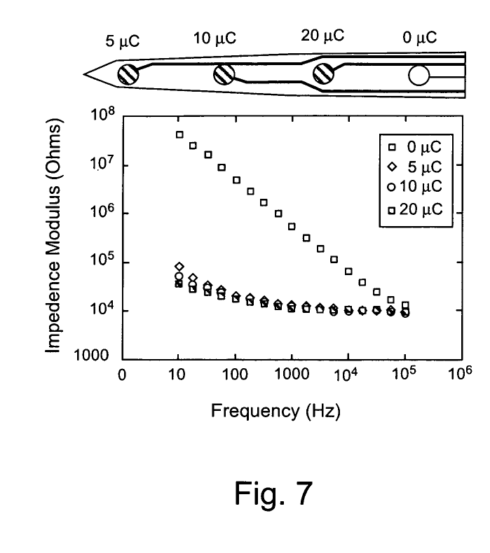

In the case of coating in vivo multielectrode arrays such as the probes of NeuroNexus Technology, the electrical properties of the film were evaluated using impedance measurements that are routinely used in neural electrode analysis for characterizing electrode/tissue or electrode/solution interfaces. As shown in FIG. 7, in one study three electrodes of a probe were coated with different amounts of PEDOT (black film represented by cross-hatching) using deposition charges of 5, 10 and 20 μC. The PEDOT coating significantly lowered the gold electrode impedance in the frequency range relevant to neural recording. Low impedance appears to be a common phenomena in conducting polymer-coated electrodes as a result of the higher surface area and more efficient charge transport of the polymer coating. This is important because having low impedance is beneficial in recording minute extracellular neural signals. The polymer electrode also has the capacity of being modified to include species that will either promote neuronal growth or inhibit glial scaring to combat the problem of chronic neural electrode/tissue interface⁻. Acute and chronic recordings using surface modified probes from rat cortex are, for example, routinely conducted.

In the above studies, conductive polymer-based drug release systems were integrated into macroelectrodes and into two types of multielectrode arrays: a Panasonic MED64 for in vitro studies; and a neural probe available from NeuroNexus Technology for in vivo recordings. The same approach can be readily applied to other types of multielectrode arrays and to metal or semiconductor electrodes. Controlled release systems can, for example, be readily prepared for numerous neurochemicals that cover a full range of excitatory and inhibitory factors.

As described above, we have electrochemically incorporated AP5 on polypyrrole coated macroelectrodes and released them in their bioactive form. Furthermore, we have incorporated CNQX and AP5 into polypyrrole coatings on the microelectrodes of MED64 and triggered CNQX release with electrical pulses and seen local inhibition of neural activity. The chemical structures of those molecules and the excitatory neurotransmitters glutamate and acetylcholine are shown below.

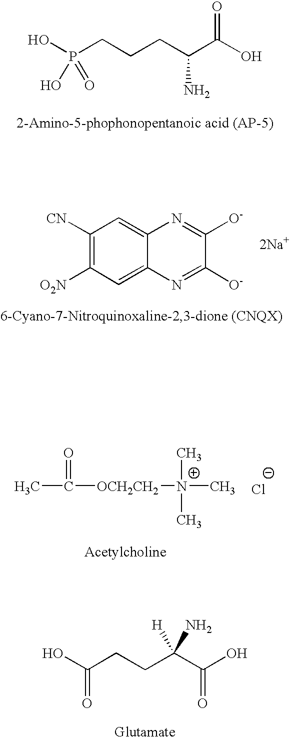

The effects of these agents are relatively easy to measure and they can dramatically influence neuronal circuit dynamics. They are relatively small molecules with negative or positive charges in which the drug loading and releasing mechanisms are described by the mechanisms set forth below.

In that regard, both anionic and cationic agents can, for example, be incorporated into and electrically released from conducting polymer electrodes. Anionic and cationic agents require different loading and release mechanisms. Embodiments of a general mechanism for drug release for anionic and cationic chemical agents are shown below:

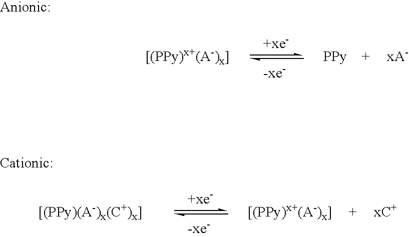

Anionic drugs can act as dopant molecules that are incorporated into the polymer by electropolymerization. When polymer/drug complex is under a negative potential, the positively charged polymer backbone is reduced and loses charge; as a result, the drug molecules dissociate from the backbone and diffuse out of the film and into the electrolyte solution. AP-5, CNQX and glutamate are negatively charged molecules, so their release systems utilize this mechanism. For cationic delivery, an anionic polyelectrolyte (specifically, poly-styrenesulfonate, a commonly used polyelectrolyte dopant for conducting polymers) will be added. Some of its negative charge will balance the charge on the polypyrrole backbone, while the extra negative charge can be used to bind to the cationic drug. Drug molecules can be loaded during the electropolymerization or after polymerization when the polymer is soaked in drug solution under negative electrical potential. The release of the cationic drug also utilizes the redox property of the polymer, but with positive potential as the trigger. The acetylcholine release system can use this mechanism.

Non-ionic or neutral molecules can, for example, be incorporated within the conductive polymers of the present invention by first functionalizing such molecules so that they are charged. For example, the neutral molecule or chemical agent can be associated with a charged molecule for incorporation into the conductive polymers of the present invention. Further, a neutral or charged chemical agent can be encompassed, encapsulated or entrapped within a conductive polymer without ionic or other forces or bonding. The entrapped compound can, for example, be released upon conformational or other changes within the polymer induced by electrical cycling.

In the studies set forth above, the electrode devices, systems and methods of the present invention were not fully optimized. Several properties of polymer-coated electrodes for chemical agents can be examined in optimizing the devices, systems and methods of the present invention. Important properties include: (1) drug-loading capacity; (2) drug-releasing efficiency (speed and consistency); (3) electrical impedance of coated electrodes; (4) chemical and electrical stability of the polymer-drug complex. Electrodeposition conditions (e.g., concentration of the monomer and dopants, current density and charge density) and electrical stimulation parameters (e.g., amplitude of the potential, potential waveform, speed and range of the potential scan, etc.) have been found to influence the drug loading and release efficiency. These factors can readily be optimized via routine investigation for obtaining fast and efficient drug release and stable coating for a specific system.

Characterization experiments can, for example, be performed with macroelectrodes, which can, for example, be gold coated coverslips (0.77 cm²). Parameters (that can affect coating/releasing properties) to be optimized can, for example, include (1) electrochemical polymerization conditions such as drug concentration and deposition charge density; (2) film thickness and porosity; (3) electrical voltage profile and amplitude used for triggering drug release. Using macroelectrodes, iterative experiments of characterization can be performed routinely and quickly and the above parameters can readily be optimized by those skilled in the art for each candidate chemical agent/drug molecule. Once optimized coating and drug-release is achieved in macroelectrodes, one can readily scale down the system to microelectrodes on, for example, MED chips as described above, and perform further characterization and experiments as described below. Parameters to study in system optimization include the (minimal) current amplitude and duration for efficient drug release from the microelectrodes that will have significant biological effects on nearby neurons cultured on the chips. AP-5 and CNQX have, for example, been studied in macro- and microelectrodes.

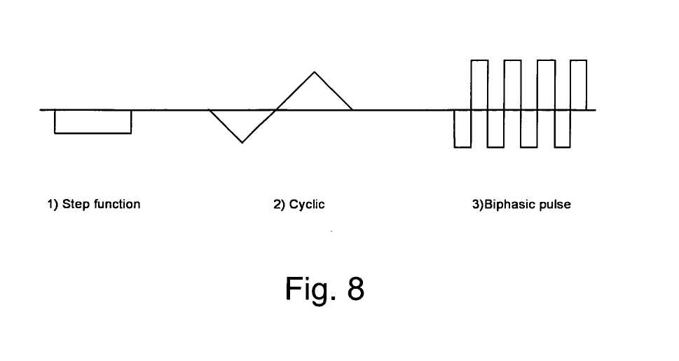

Electrodeposition conditions for drug loading and release can first be studied using macroelectrodes as described above. Conductive polymer-drug (for example, PPy-drug or PEDOT-drug) complexes can be electrically deposited on the gold surface, and the coated electrode can then be immersed in 1 ml of PBS solution and subjected to various electrical release stimuli as, for example, illustrated in FIG. 8.

After each stimulus, the concentration of released drug in the solution can be quantified using different analytical tools as known in the art. For example, CNQX has a characteristic UV absorption band at 254 nm, and can thus be quantified by UV spectrometer. For AP5, OPA (o-phthaldialdehyde) derivatization can be performed and fluorescence and can be used to quantify the released molecules. For glutamate and acetylcholine, commercial enzyme-based fluorometric assays are readily available. For each drug being tested, this large scale release can be used to characterize and optimize the release characteristics. Macroelectrodes provide an inexpensive yet efficient system to test gross coating and release protocols. The larger surface area of macroelectrodes allows for easier quantification of drug loading and release. Use of macroelectrodes is, therefore, a cost-effective method allowing us to rapidly complete many initial screenings and tests before coating microelectrodes and testing them with neuronal cultures.

In addition to the release profile, electrical impedance of the electrode coated with the conducting polymer-drug film before and after drug release can be characterized using impedance spectroscopy. Further, electrical, chemical and mechanical stability of the polymer-drug film in the culture media, before and after repetitive release, can be characterized using cyclic voltammetry, Fourier Transform Infrared Spectroscopy, and surface analyses such as optical microscopy, scanning electron microscopy and atomic force microscopy.

In the case of microelectrodes (for example, of the MED64 System), the drug release systems developed/optimized for macro-electrodes can be scaled down to the micro-scale and applied on the microelectrodes. The hardware and software connections of the studies described above allow the electrochemistry to be done in, for example, the MED64 dish on individual electrodes. Optimal electropolymerization conditions determined with macro-electrodes can be used as starting conditions for such microelectrodes. The electrochemical properties of the coating (impedance and redox activity) can be characterized and optimized for microelectrodes. To characterize the drug-release properties of these electrodes for the candidate drugs, the biological responses of living cells (for example, neurons grown near the electrodes) can be studied as described in connection with the studies of FIGS. 6A through 6E above. Whole cell patch clamp recording can be used to measure the responses of neurons to the released drug molecules and thereby characterize the amount and time-course of release under different voltage pulse amplitudes and durations. The effect of changing the distance between the drug-releasing electrodes and the recorded cells can also be studied to estimate the effective diffusion time and range of the released agent.

For patch clamp recordings, low density neuronal culture (1000 to 3000 cells per electrode area of the MED64 probe) can be used. Hippocampal neurons and glial cells can, for example, be cultured on the MED64 surface, with the microelectrode array pre-coated with conducting polymer loaded with one or more candidate chemical agents. Perforated patch-clamp recordings can be made on a selected neuron. For agents such as glutamate that can activate specific membrane conductance, any recorded neuron can serve as a sensor. Once can then stimulate specific electrodes with short voltage pulses (for example, in the range or approximately −0.3 to −0.8 V for approximately 0.1 to 10 s) to release glutamate. Whole-cell current recorded in voltage-clamp mode can be used to characterize stimulated drug release and determine the diffusion profile. As a control, the response to glutamate release from the electrodes can be compared to the response of the same neurons to brief picospritzer puffs of fixed volumes of glutamate-containing solution. Similar experiments can, for example, be done for acetylcholine.

For an agent such as CNQX or AP-5, which inhibits a specific membrane conductance, one must first activate the conductance to measure the drug's effects. Activating conductance can be done by stimulating the recorded neuron if it has a glutamatergic autaptic connection (that is, a connection to itself, which is commonly found in culture). To test drug release, a loaded microelectrode can be stimulated first, followed by cellular stimulation that activates synaptic currents. Presence of the drug will cause a decrease in synaptic current compared to control conditions if a sufficient concentration of drug has diffused to the synaptic sites.

The devices, systems and methods of the present invention can, for example, be used to evaluate the effectiveness of transient local drug-release on perturbing neuronal network activity in vitro. The effects of controlled drug-release systems on the spatio-temporal pattern of spontaneous bursting activity in neuronal networks in vitro can, for example, be studied. Such studies can be used to test the combined power of microelectrode array recording with controlled local drug-release in neurobiological applications.

In high density cultures of cortical neurons (for example, 20,000 in the well), cells begin to form connections by approximately 48 hrs. By 3 days in vitro or DIV, spiking activity can be observed through the integrated extracellular microelectrodes of the MED64 System. Spike sorting with Offline Sorter ( available from Plexon Inc. of Dallas, Tex.) shows that we record from 150 to 200 neurons at once. For these sets of experiments, the majority of the electrodes in the MED can be coated with conducting polymer/neurochemicals. The remaining electrodes can be either uncoated (platinum black) or coated with polymer without drugs to serve as controls to evaluate the effect of the electrical stimuli alone. As described above, drug release is triggered by electrical stimuli, which can possibly depolarize the neurons or simply induce certain electrochemical reaction to affect the culture. Preliminary studies have shown that high cathodic simulation (for example, greater than 3V) can damage the electrode and/or nearby cells which results in loss of neural activity in the recording. Such stimulus should be avoided. With milder conditions (for example, less 3.0V), we have not found any observable neural activity change. Therefore, the effects that have been observed are the results of the effect(s) of the released chemical agent(s). Since new forms of stimuli may be used for different drugs, such controls should always be included.

To perturb network activity, we will choose networks that exhibit robust spontaneous spiking activity registered at multiple electrodes. Following a period of baseline recording, we will stimulate one or more pre-selected drug-loaded electrodes using previously optimized protocols for a brief period of time, and then record (perturbed) network activity until it recovers to a normal level. Depending on the response, multiple trials will be done and the averaged peristimulus (i.e. peri-drug-release) time histogram of spiking activity at all channels will be analyzed to find out the range and duration of the drug effect. To determine the effective life-time of the release system, we will continuously run the same trials on the same electrodes until the effect diminishes (i.e. when drug is exhausted). This will provide useful information for chronic applications.

In vivo studies can, for example, include incorporation of drug-polymer complex onto an in vivo multielectrode array system such as acute or chronic probes available from NeuroNexus Technology. The modified microelectrode arrays can, for example, be implanted into rat cortex to study controlled drug release. Acute and chronic implantation studies can be performed.

The electrodes can, for example, be studied by recording activity of single units and local field potentials from somatosensory cortex in response to periodical stimulation of the large facial vibrissae (the “whisker/barrel” system). One can apply various voltage pulses to the electrodes to trigger drug release at specific sites. Effects of drug release can, for example, be assessed based on subsequent changes in spontaneous and/or sensory-evoked activity. It is expected, for example, that CNQX and AP-5 release will reduce the firing rates recorded at the same and at nearby electrodes, whereas glutamate and acetylcholine release could enhance neuronal activity. Based on the response and the electrode configuration, we can estimate the speed and range of effective drug diffusion (with consideration of neuronal circuit mechanisms). The maximal number of release stimuli that can trigger a perceivable effect can also be determined, from which the capacity of drug supply of the system can be estimated.

The diffusion of a drug in vivo could be different from the more open in vitro environment. Therefore, more drug may need to be released to cause detectable effect on neural activity at the same neuron-electrode distance. One goal is to determine how many times the electrode array can release enough drug molecules to affect network activity. If the rounds of effective release (defined as Nₐ) is sufficiently high, it will be feasible to extend the same method in the future for some long-term applications. If the effective release rounds are too few, it may be necessary to develop specialized electrodes of larger surface area to increase the loading capacity as discussed below.

There are perceivable challenges in chronic implantations such as high demands for drug load and high diffusion barriers caused by host tissue reaction. A drug release system such as the conductive polymer-CNQX system described above can, for example, be used a representative system to evaluate the effect of host tissue upon the drug release profile. The findings from one drug release system should apply to other drugs.

CNQX loaded chronic neural probes can, for example, be implanted in barrel cortex of rats as described above. The probe and chronic connector can be anchored on the skull and the opening can be sealed with dental acrylics. After implantation, the activity of single units and local field potentials from somatosensory cortex in response to periodical stimulation of the large facial vibrissae (the “whisker/barrel” system) as described above can be recorded daily. Once we verify that one electrode is still functional in recording evoked signals, we can trigger the drug release at this electrode using the effective stimuli from acute experiments to evaluate the drug effect on the same and/or neighboring electrodes. Over time, it may be necessary to increase the number or duration of the stimuli to compensate for drug depletion as well as increased diffusion barrier created by the host tissue reaction. Such experiments can be continued with various test frequencies over days until drug effect diminishes or the probe fails to record. In the end, the total number of effective drug-release trials (summed as N_{c}) will be compared to Nₐ as determined above. If Nₐ˜N_{c} (and is sufficiently large), we would have efficiently used the drug in a chronic situation and the host tissue reaction is not a major factor. If Nₐ>N_{c}, the host tissue might play a role either by degrading the polymer coating or by fouling and encapsulating the electrode or neuronal retraction. The exact effect of the host tissue response can be evaluated at the end points.

At the end points, the animals can be anesthetized and perfused following IACUC regulation guidelines. The brain can be isolated, fixed and sliced for immunohistological procedures. The tissue slices can be immunostained with GFAP antibody for astrocytes, ED1 and Iba-1 antibodies for microglia and macrophages, and anti-neurofilament for neurons. The staining pattern will reveal both qualitatively and quantitatively the degree of glial sheath formation, as well as the composition of cellular population around the implant. The probes will be removed from the brain and also stained to examine protein and tissue deposition. The polymer coating will be examined under SEM for its mechanical integrity. It is also possible to keep the electrical connection active and further stimulate the probe in small volumes of buffer solution to examine if there is still significant amount of drug molecules remaining in the polymer.

If the results of the studies indicate that the studied drug release design provides too few doses of drug to evaluate long term effect, the drug load can be increased for further chronic applications. Increasing the polymer-drug film thickness can increase the drug load. However, based on our experience, a thicker film does not necessarily result in improved drug release, probably because of the diffusion barrier for the drugs within the inner side of the film. In addition, thicker films have a higher tendency to detach from the electrode substrate. A straightforward and simple approach to increase drug load is to increase the electrode surface area, which can, for example, be done by roughening the electrode area with electrodeposited gold or platinum black. Studies have shown that roughening electrodes of the probes by electroplating with gold can result in an 8 fold increase of the effective surface area for polymer coating. See, for example, Cui, X. and Martin, D. C., Fuzzy gold electrodes for lowering impedance and improving adhesion with electrodeposited conducting polymer films. Sensors and Actuators A: Physical, 103(3), 384-394 (2003). In addition, the roughened substrate promotes the adhesion between the polymer and the electrode, which is desired for long term application. Platinum black is the electrode material of MED64 and has even higher surface area and supported the growth of polymer-drug coating very well. Pt black can also be electroplated on in vivo probes available from NeuroNexus Technology before the polymer coating. If further increase of surface area is required, larger electrodes can designed and be used on the probe for the drug release, while the remaining electrodes can be used to record and to evaluate the drug effect from a distance.

The foregoing description and accompanying drawings set forth the preferred embodiments of the invention at the present time. Various modifications, additions and alternative designs will, of course, become apparent to those skilled in the art in light of the foregoing teachings without departing from the scope of the invention. The scope of the invention is indicated by the following claims rather than by the foregoing description. All changes and variations that fall within the meaning and range of equivalency of the claims are to be embraced within their scope.

What is claimed is:

1. An electrode system comprising at least one electrically conductive element to effect at least one of delivery an electrical signal to living cells in the vicinity of the electrically conductive element or measurement of an electrical signal from living cells in the vicinity of the electrically conductive element, the electrically conductive element comprising a first conductive polymer, the first conductive polymer comprising at least a first chemical agent to affect activity of the living cells, the first chemical agent being releasable from the first conductive polymer to interact with the living cells upon application of a first electrical potential to the electrically conductive element.

2. The electrode system of claim 1 wherein the first conductive polymer is polypyrole, poly(3,4-ethylenedioxythiophene), polyaniline, polythiophene, poly(acetylene), poly(fluorene), polytetrathiafulvalene, polynaphthalene, poly(p-phenylene sulfide), poly(para-phenylene vinylene), a derivative of the above, a copolymer of the above, or a mixture of the above.

3. The electrode system of claim 1 wherein the first conductive polymer is deposited upon the electrically conductive element via electropolymerization.

4. The electrode system of claim 1 wherein the living cells are neuronal cells and the first chemical agent is a first neurochemical.

5. The electrode system of claim 5 wherein the first neurochemical is a neurotransmitter, a neuromodulator, an agonist or a receptor antagonist.

6. The electrode system of claim 4 wherein the first neurochemical is 6-cyano-7-nitroquinoxaline-2,3-dione, 2-amino-5-phosphonopentanoic acid, glutamate, acetylcholine, epinephrine, norepinephrine, dopamine, serotonin and melatonin, glutamic acid, gamma aminobutyric acid (GABA), aspartic acid, glycine, adenosine, adenosine-5′-triphosphate (ATP), guanosine triphosphate (GTP), bicuculin, dizocilpine (MK-801), or a derivative of one of the above.

7. The electrode system of claim 5 comprising a plurality of conductive electrode elements, at least two of the conductive electrode elements comprising the first conductive polymer and the first neurochemical.

8. The electrode system of claim 5 wherein the electrode system comprises at least a second conductive electrode element comprising a second conductive polymer, the second conductive polymer comprising a second neurochemical that is releasable upon application of a second potential to the second conductive electrode.

9. The electrode system of claim 8 wherein the first conductive polymer and the second conductive polymer are the same, the first neurochemical and the second neurochemical are the same and the first potential and the second potential are the same.

10. The electrode system of claim 8 wherein the first neurochemical and the second neurochemical are different.

11. The electrode system of claim 10 wherein the first conductive polymer and the second conductive polymer are different.

12. The electrode system of claim 8 wherein the first potential and the second potential are different.

13. The electrode system of claim 8 wherein the electrode system is a multielectrode array comprising a plurality of electrically conductive microelectrode elements.

14. The electrode system of claim 13 wherein the first conductive polymer and the second conductive polymer are selectively deposited upon electrically conductive elements of the multielectrode array via electropolymerization.

15. The electrode system of claim 14 wherein the chemical agent is repeatably releasable from the first conductive polymer to interact with the living cells upon cyclic application of the first electrical potential to the electrically conductive element.

16. A method of applying a chemical agent to living cells, comprising: contacting at least one electrically conductive element of an electrode system with the living cells, the electrically conductive element being adapted to effect at least one of delivery an electrical signal to living cells in the vicinity of the electrically conductive element or measurement of an electrical signal from living cells in the vicinity of the electrically conductive element, the electrically conductive element comprising a first conductive polymer, the first conductive polymer comprising at least a first chemical agent to affect activity of the living cells, and causing the first chemical agent to be released from the first conductive polymer to interact with the living cells by application of a first electrical potential to the electrically conductive element.

17. The method of claim 16 wherein the first chemical agent is a first neurochemical.

18. A method of fabricating an electrode system comprising at least one electrically conductive element to effect at least one of delivery an electrical signal to living cells in the vicinity of the electrically conductive element or measurement of an electrical signal from living cells in the vicinity of the electrically conductive element, comprising: applying at least a first conductive polymer, the first conductive polymer to the electrically conductive element, and doping the first conductive polymer with at least a first chemical agent to affect activity of the living cells, the first chemical agent being releasable from the first conductive polymer to interact with the living cells upon application of a first electrical potential to the electrically conductive element.
